(19) United States
(12) Patent Application Publication
(10) Pub. No.: US 2014/0171793 A1
(43) Pub. Date: Jun. 19, 2014

(54) METHODS FOR DETECTING AND TRACKING NEEDLE

(71) Applicants: Feng Lin, Wu Xi (CN); Christopher Hazard, Niskayuna, NY (US); Seyed-Bolorforosh Mirsaid, Guilderland, NY (US)

(72) Inventors: Feng Lin, Wu Xi (CN); Christopher Hazard, Niskayuna, NY (US); Seyed-Bolorforosh Mirsaid, Guilderland, NY (US)

(21) Appl. No.: 14/241,677

(22) PCT Filed: Aug. 31, 2012
(86) PCT No.: PCT/US12/53369

§ 371 Date: Feb. 27, 2014

(30) Foreign Application Priority Data
Aug. 31, 2011 (CN) 201110289172.1

(51) Int. Cl. A61B 8/08

(52) U.S. Cl. CPC A61B 8/0841; A61B 8/5276
USPC 600/424

(57) ABSTRACT

A method for detecting a needle, the method comprises arranging an ultrasound probe such that it scans an area covering a needle inserted into a tissue and nearby tissue around the needle, collecting a plurality of ultrasound frames associated with motion of the nearby tissue, determining movement information of the nearby tissue, post-processing the movement information of the nearby tissue to determine a position of the needle, and outputting information relating to the position of the needle.

### BACKGROUND OF THE INVENTION

1. Field of the Invention

Embodiments of the present invention relate to ultrasound imaging, and more particularly to methods for detecting and tracking a needle.

2. Description of the Prior Art

When an abnormal tissue, for example, tumor is observed non-invasively, it is generally necessary to study and diagnose that tissue to determine a proper therapy. This necessitates removal of a sufficient sample of the tissue from the body of a patient for pharmacological analysis. The tissue sample may be acquired by, for example, surgical ablation, and needle puncture based biopsy. In addition to the biopsy, it is also feasible to use a needle to inject medicaments for local anesthesia and relevant treatment.

Ultrasound imaging helps to secure a needle to a desired position of the body. For example, in order to perform biopsy on the collected sample, it is fundamentally important to accurately position the needle such that the sharpened point of the needle penetrates the sampled tissue. Subsequently, the biopsy needle is tracked through an ultrasound imaging system, and guided through the target tissue to the desired depth.

However, the existing ultrasound-guided biopsy suffers from the same difficulty in detecting the needle. This is generally because the needle has a small size and the needle is tilted relative to the direction of the ultrasonic waves. Consequently, the ultrasonic waves are reflected in all directions and can hardly be received by an ultrasound probe. Besides, in the conventional 2D imaging modes, the needle tends to out of the imaging plane and thus cannot be captured by the ultrasonic wave array.

So far, a number of methods and devices have been proposed to enhance the visualization of the needle. Spatial compounding imaging helps to increase the visibility of the needle, because images are collected at different angles closer to a right angle relative to the needle such that stronger signals may be obtained from the needle. A needle with a larger tip is used such that it reflects ultrasound at a larger angle. The needle is attached to a vibrator and detects the needle vibration using a Doppler method so as to locate the needle.

Unfortunately, the aforesaid techniques still have serious drawbacks. Some of these devices are rather complicated in structure and thus lead to higher cost in manufacture; while others are hard to be operated. What is more important, all of these techniques study the needle itself. Consequently, they will not be able to detect the needle when the needle slightly out of the imaging plane. Therefore, there exists an urgent need in a technique for needle detection, which is easy to use and reliably tracks the needle even when the needle slightly falls off the imaging plane.

### BRIEF DESCRIPTION OF THE INVENTION

An embodiment relates to a method for detecting a needle. The method comprises arranging an ultrasound probe such that the ultrasound probe scans an area covering a needle inserted into a tissue and nearby tissue around the needle. The method further comprises collecting a plurality of ultrasound frames associated with motion of the nearby tissue, determining movement information of the nearby tissue, post-processing the movement information of the nearby tissue to determine a position of the needle, and outputting information relating to the position of the needle.

Another embodiment relates to a method for tracking a needle. The method comprises arranging an ultrasound probe such that the ultrasound probe scans an area covering a needle inserted into a tissue and nearby tissue around the needle. The method further comprises collecting a plurality of ultrasound frames associated with motion of the nearby tissue, and determining movement information of the nearby tissue. The method further comprises post-processing the movement information of the nearby tissue to determine a position of the needle, and storing information relating to the position of the needle as a reference. The method further comprises determining whether current speckle data is correlated to the reference using speckle tracking, determining the position of the needle to be lost if the current speckle data is not correlated to the reference, and determining the position of the needle to remain valid if the current speckle data is correlated to the reference.

### BRIEF DESCRIPTION OF THE DRAWINGS

Embodiments of the present invention will be more apparent upon description with reference to the following drawings, in which:

FIG. 1 is a sectional view of the needle and the nearby tissue used in the needle detection method according to an embodiment of the present invention;

FIG. 2 is a block diagram of the needle detection method of FIG. 1 according to an embodiment of the present invention;

FIGS. 3A and 3B respectively illustrate the displacement and strain of the nearby tissue according to an embodiment of the present invention;

FIG. 4 illustrates a post-processing according to an embodiment of the present invention;

FIG. 5 illustrates the displacement being mapped to the gray level according to an embodiment of the present invention;

FIG. 6 illustrates a directional smoothing processing according to an embodiment of the present invention;

FIG. 7 illustrates an ultrasound image obtained when the needle remains on the imaging plane and the corresponding tissue strain according to an embodiment of the present invention;

FIG. 8 illustrates an ultrasound image obtained when the needle slightly out of the imaging plane and the corresponding tissue strain according to an embodiment of the present invention; and

FIG. 9 illustrates a flow diagram of a needle tracking method according to an embodiment of the present invention.

### DETAILED DESCRIPTION OF THE INVENTION

Hereunder the technical solutions according to embodiments of the present invention will be described at length with reference to the drawings of the present invention. Obviously, the embodiments to be introduced below should not be construed to be restrictive to the scope, but illustrative only. Persons skilled in the art would understand that other embodiments that are obtained based on the present invention without exercising inventive skills also fall into the scope of the present invention.

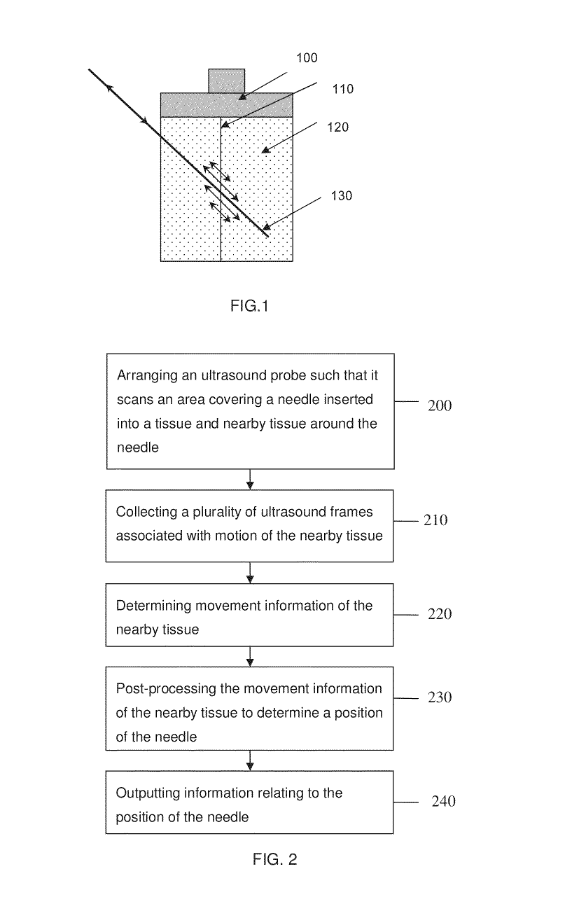

FIG. 1 is a sectional view of the needle and the nearby tissue used in the needle detection method according to embodiments of the present invention. By “nearby tissue”, the term is intended to be representative of the tissue around the needle that is inserted into the body of interest. The tissue includes a tissue from the body of a human being or the body of an animal, which also includes liquid and gas inside the body. As is shown in FIG. 1, reference numeral 130 denotes a needle which is inserted into the tissue in a direction tilted from an ultrasound beam 110 emitted from an ultrasound probe 100. Since the biopsy needle is usually rather small, direct measurements of the echoes reflected from the needle would be rather challenging, and moreover have been proved unreliable and inaccurate. Consequently, a method according to an embodiment of the present invention studies not only the dynamics of the needle itself but also the dynamics of the nearby tissue 120. As shown in FIG. 1, the bidirectional arrows represent the back and forth movements of the needle along the length direction of the needle. To be more specific, they represent the movements of the nearby tissue caused by the displacement of the needle. The “length direction of the needle” refers to the direction along which the needle body extends. The dynamics of the needle and the nearby tissue are the subjects to be studied in the present invention. Although FIG. 1 shows that the needle is moved along the length direction, this should not be construed to be restrictive to the scope of the present invention. Persons skilled in the art would understand that this is simply one possibility of the needle movements. For example, the biopsy needle may rotate around the position where the needle is inserted.

According to the physical friction principle, the nearby tissue will move along with the moving needle. The closer to the needle, the longer distance the tissue travels. In an embodiment of the present invention, a hand may move the needle back and forth along the length direction such that the nearby tissue moves with the needle. According to an embodiment of the invention, a vibrator may be used to cause the needle to move back and forth along the length direction such that the nearby tissue moves with the needle. The vibrator may have a conventional structure and design, and therefore is not introduced in detail here.

The needle detection method according to an embodiment of the present invention is described in details below with reference to FIG. 2. As shown in FIG. 2, the needle detection method comprises the following steps: arranging an ultrasound probe such that it scans an area covering a needle inserted into a tissue and nearby tissue around the needle (at Step 200), collecting a plurality of ultrasound frames associated with motion of the nearby tissue (at Step 210), determining movement information of the nearby tissue (at Step 220), post-processing the movement information of the nearby tissue to determine a position of the needle (at Step 230), and outputting information relating to the position of the needle (at Step 240). At Step 200 an ultrasound probe is positioned such that it scans an area that covers a needle inserted into a tissue and the nearby tissue around the needle. According to an embodiment of the present invention, the ultrasound imaging may be B-mode ultrasound imaging, which displays organs as well as the position, dimension, shape and echoes of pathology at different grays as a two-dimensional white and black image, so as to provide information regarding the pathology or lesions for clinical purpose. Subsequently, a hand or a vibrator is used to cause the needle to move, for example, back and forth along the length of the needle as well as causing the nearby tissue to move along with the moving needle. At step 210, a plurality of data frames associated with the ultrasound echoes are collected while the nearby tissue moves.

Then, an analysis is conducted at Step 220 on the collected data frames of the ultrasound echoes to determine the displacement or strain of the nearby tissue. In an embodiment of the present invention, a speckle tracking method is used to determine the displacement or strain of the nearby tissue. In particular, the speckle tracking is performed between the data frames to determine the displacement or strain of the nearby tissue. Speckle tracking is widely used in ultrasound image analysis applications, like elasticity, registration, and motion correction. Compared to the Doppler method that is commonly used for flow measurement, the speckle tracking method is more sensitive to small motion (accurate to sub microns), is better suitable to slow motions, has better resolution, and requires only two data sets (packet size of two) for calculation. Speckle tracking is suitable for needle detection for the above-mentioned reasons.

Speckle tracking is a new technique that is developed from strain and strain rate imaging. An ultrasound image is consisting of numerous small pixels, i.e., natural acoustic markers. They are stable acoustic speckles uniformly distributed among the tissue around the biopsy needle, and move with the tissue in synchronization and do not obviously change in shape between consecutive frames. The speckle tracking imaging tracks each speckle consecutively frame to frame, and computes the moving track of each speckle, thereby quantitatively displaying the displacement and strain of the tissue. Strain is defined as change in the dimension of the tissue under an application of force, and may be derived from displacement data of the corresponding, local nearby tissue.

Either of the beamformed RF data, demodulated RF data, or the detected amplitude data may be used as input of the speckle tracking method. RF data may provide more accurate results than the amplitude data because RF data may be more computationally intensive and contain phase information. Speckle tracking may be implemented using one of the following algorithms: 1D or 2D cross correlation and the derivations, phase-based iterative methods, and optical flow etc. Either the displacement or strain (derivative of displacement) may be estimated through speckle tracking.

As is described above, although the speckle tracking is implemented in an embodiment of the present invention, persons skilled in the art would understand that the movement information associated with the nearby tissue, for example, displacement or strain of the nearby tissue, may be also obtained using a Doppler method. The method for detecting a needle using the Doppler method comprises the following steps: arranging an ultrasound probe such that it scans an area that covers a needle inserted into a tissue and nearby tissue; collecting a plurality of ultrasound frames associated with motion of the nearby tissue; determining movement information between frames using the Doppler method; post-processing the movement information of the nearby tissue to determine the position of the needle; and outputting information relating to the position of the needle. Given the present disclosure, persons skilled in the art would understand, based on the existing knowledge of the Doppler imaging, how to detect a needle by tracking the movement of the nearby tissue using the Doppler method. Thus details are omitted here.

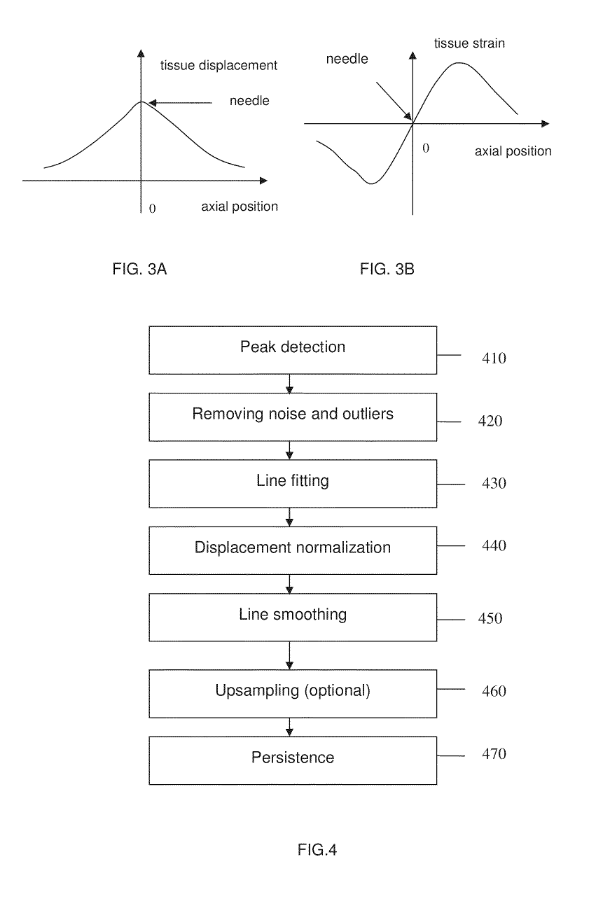

So far, the displacement or strain of the tissue around the needle has been obtained using the speckle tracking or Doppler method, which may subsequently be analyzed to roughly estimate the position of the needle. Hereunder FIGS. 3A and 3B are relied on to explain the displacement and strain of the tissue (the axial component) along the direction of the ultrasound beam.

FIGS. 3A and 3B respectively illustrate the displacement and strain of the tissue (along the y-axis) being mapped onto the axial position (along the x-axis). As is discussed above, the closer to the needle, the greater distance the tissue travels. Axial position of 0 is at the position where the beam crosses over the needle. The tissue displacement is maximal at the needle and the signs of the strain are opposite at sides of the needle. This property can be used to accurately estimate the needle position.

In particular, a frame of displacement/strain data (a 2D data frame as a function of axial position and ultrasound beam) may be obtained first. An analysis algorithm will then be performed on the frames. The analysis algorithm first detects if there is outstanding motion along a line compared to the background and if the line is positioned and oriented reasonably like a needle. If so, the needle position may be estimated from the 2D data frame based on the maximum of displacement or boundary between positive and negative strains.

Subsequently, the image data concerning the tissue displacement or strain obtained above may be subjected to the post-processing illustrated in FIG. 4. After the post-processing, the raw estimation of the needle position may be further smoothed or fit into a line or curve to more accurately determine the position of the needle.

FIG. 4 shows a flow diagram of the post-processing. The purpose of the post-processing is to obtain the information or image of the needle position from the image data concerning the tissue displacement or strain. FIG. 4 only illustrates one of the implementations of the post-processing. The displacement data frames are the input data in this embodiment. The post-processing comprises the following steps: peak detection (at Step 410); removing noise and outliers (at Step 420); line fitting (at step 430); displacement normalization (at Step 440); line smoothing (at Step 450); upsampling (at Step 460); and performing persistence (at Step 470).

The post-processing starts from detecting the peak at step 410. For each beam in a frame, a peak position is detected by identifying the maximal location of the absolute value of the displacement. To get sub-sample resolution, interpolation or other known methods may be applied.

Subsequently, noise and outliers are removed at step 420. Since not every beam contains needle information, a smart algorithm is designed to exclude beams that are not likely to contain needle information in order not to affect the accuracy of the subsequent line fitting in Step 430. A noise beam is a beam with a displacement curve that does not have an obvious peak. For example, the curve is up and down with multiple peaks, or the peak value is not significantly higher than average displacement along the curve. An outlier beam is a beam with a peak position that is significantly different from peak position of nearby beams with valid needle information. The outliers may be caused by false calculation of displacement or needle detection. The noise and outlier beams haven been excluded after step 420.

The detected needle peaks shall appear in the image as a straight line or a line with slight curvature. First-order line fitting or second-order line fitting may be used to model the peak positions into a line or curve. The line fitting may be implemented at step 430 by Hough transformation or linear regression that is well-known to the domain expert.

Following the line fitting step is displacement normalization at step 440. A raw needle image is formed by the group of needle peaks obtained at Step 420 with value of each peak as the displacement value. A soft threshold is defined. As is shown in FIG. 5, the soft threshold may be defined as a certain ratio of the maximal displacement, for example, 50% of maximal displacement. The peak value is remapped to a range of gray scale based on the threshold. The range of gray scale may be, for example, from 0 to 255. The mapping may be a linear mapping as shown in FIG. 5. In the example, a displacement value at the threshold is mapped to gray scale of 0, and the maximal displacement “Max_Disp” is mapped to a predefined gray scale of “Max_Gray”. Max_Gray determines the brightness of the needle in the display, can be defined to be, for example, 180, with a maximal gray scale of 255.

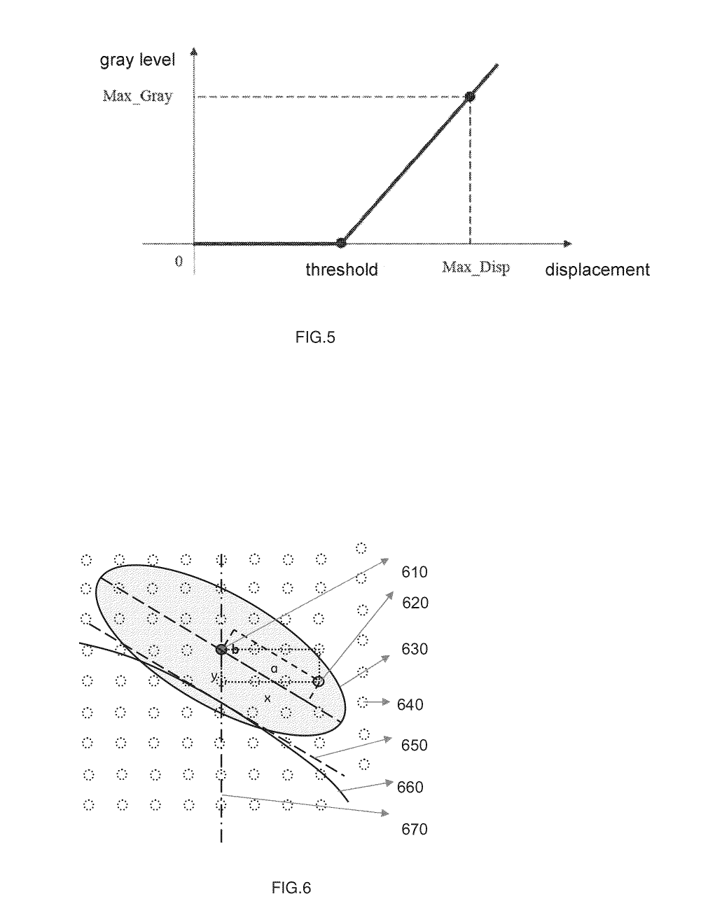

Then line smoothing is implemented at the following step 450. The needle image obtained in the last step is a group of discrete fine points. To make it more like a needle, further processing is performed. A smoothing may be applied to connect points into a line. The smoothing can be simple two-dimensional low-pass filtering. Or, to make it more sophisticated, more filtering may be performed with respect to the data along the needle direction, while less filtering when it is perpendicular to the needle. The needle direction is determined during the step of line fitting. The directional smoothing is further illustrated in FIG. 6. Reference numeral 610 denotes the sample to be smoothed; reference numeral 620 denotes a sample in the smoothing range; reference numeral 630 denotes an ellipsoid smoothing window with long axis parallel to the 1st-order line; reference numeral 640 represents an image sample grid; reference numeral 650 represents 1st order needle line fitting at the sample to be smoothed; reference numeral 660 represents 2nd order needle line fitting; and reference numeral 670 represents axial axis. Since the relevant processing methods are similar to the conventional techniques, they are not introduced in detail in the present disclosure. The effective point-spread function of the filter is a ellipsoid with a long axis along the needle direction.

Optionally, an upsampling step may be formed at step 460. The needle image may be up-sampled to higher resolution to show smoother looking. The upsampling can use linear interpolation or 2nd order interpolation that is well known.

The needle motion is dynamic and hence different frame of needle may show different level of displacement and quality of needle information. A frame averaging method will help to make the needle look more consistent. The frame averaging may be implemented at step 470 by a simple FIR or IIR filter. To improve the performance, the frame averaging may take quality of each frame into consideration. The quality of a frame may be quantified by magnitude of displacement and/or line fitting error. The quantified quality of a frame may be used as a weight to apply to weighted frame averaging.

Upon the post-processing introduced above, the image of the needle position is obtained.

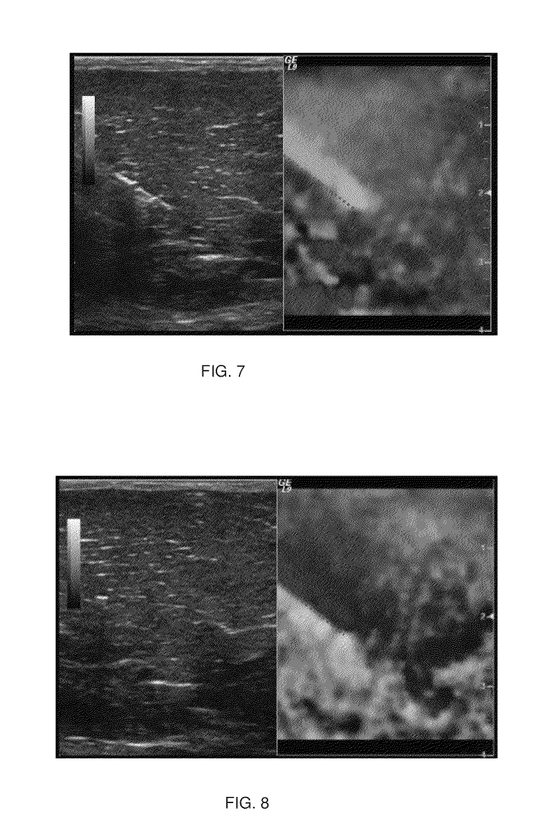

FIG. 7 shows a real ultrasound image obtained when the needle remains on the imaging plane and the corresponding tissue strain. FIG. 7 shows that a distinct line pattern shows up clearly in the strain image on the right hand side and the needle line (the dotted line) is estimated to be in between the positive and negative strain.

FIG. 8 illustrates a real ultrasound image obtained when the needle slightly out of the imaging plane and the corresponding tissue strain. The needle is not seen in the B-mode image on the left hand side due to being slightly out of plane but clearly seen in the strain image on the right hand side. As a cross-zero line is also well defined, it enables the accurate estimation of the needle position. As such, the method proposed in the present invention is able to accurately determine the position of the needle even when the needle slightly out of the imaging plane.

The speckle tracking method may be combined with the existing amplitude methods to detect the needle with more confidence or further fine-tune the needle position. This is especially the case if it is clinically required to validate the needle position by seeing the needle in the B-mode image.

The detection process may be repeated in real-time when scanning. For example, for every 0.5 second, the detection process is activated. The needle position is updated if valid motion is identified.

Turning back to FIG. 2, the image subjected to the post-processing is outputted at step 240. According to an embodiment of the present invention, the image may be displayed on a display, or may be printed on a printer in an embodiment of the present invention. The needle detection and tracking according to an embodiment of the present invention work for different needle orientations. If the needle is in the imaging plane, the tissue motion shows up as a pattern of a line. If the needle is vertical to the imaging plane, the tissue motion shows up as a pattern of a point. For 3D imaging, the needle orientation is less relevant. As such, from the perspective of persons skilled in the art, the above method can easily be extended to detect the needle in 3D space.

The needle may be displayed on top of the B-mode image as a colored semi-transparent line. Alternatively, only the needle tip is displayed if that is only point of interest. A side-by-side display mode is also an option to show image without needle on one side and image with needle line/tip on the other. There may be means to display the status and quality of the detection/tracking. The status may include: (1) No valid detection has been made, (2) A valid detection is just made, (3) Valid detection was made and currently in tracking mode, and (4) Out of correlation and needle position is lost.

The means may include different colors or different line types of the needle line, sign or text in the display, verbal warning from the scanner. The quality of the detection/tracking can be display using a meter.

For a 3D mode, the algorithm may choose to display a standard 2D view, for example, with the needle in the image plane. According to one aspect of the present invention, a stabilizer function may be arranged to lock the needle in the image when the probe is moving around.

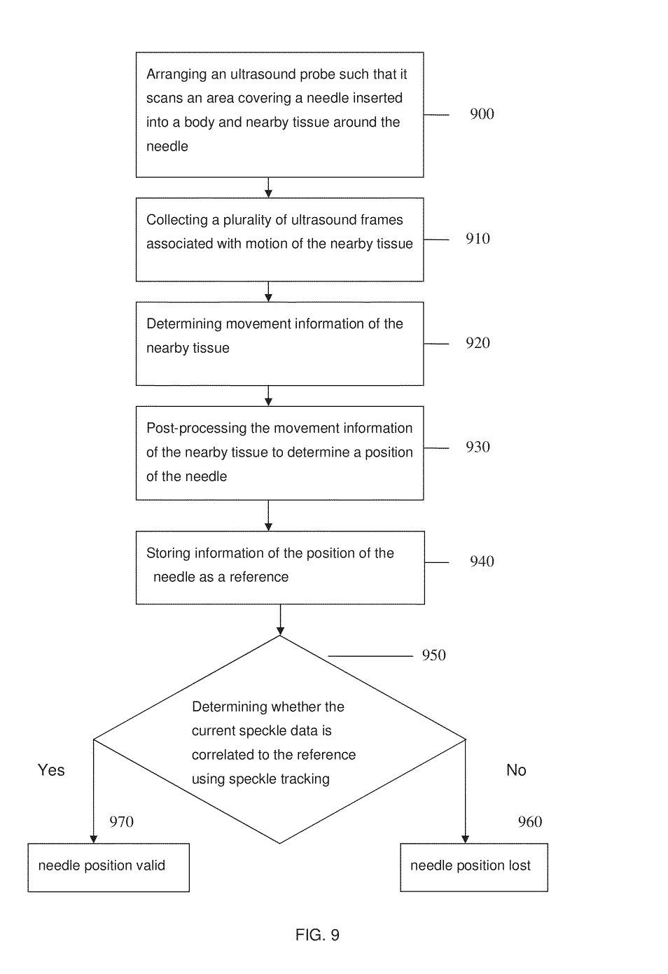

In an embodiment of the present invention, a method for tracking a needle is proposed. FIG. 9 illustrates a flow diagram of the needle tracking method according to an embodiment of the present invention. As is shown in FIG. 9, after the needle is located using the method according to an embodiment of the present invention, it is still possible to track the needle motion indirectly and determine whether the needle position is still valid by estimating tissue motion around the needle when needle stops moving. Steps 900-930 are similar to the corresponding steps as shown in FIG. 2, and thus are not detailed here. When the image data relating to the needle position is obtained at step 930, the image data may be stored as reference at step 940, which may be used for judging the position of the needle later on. As is discussed above, a typical speckle-tracking algorithm may be used to consecutively track each speckle frame after frame and compute its movement track so as to quantitatively display the displacement and strain of the tissue. If it is found that the speckle de-correlates from the reference by too much over time at step 960, the position of the needle is determined as being lost. Then, the algorithm may reset the needle position and/or ask the user to poke the needle to reinitialize the needle detection process. Otherwise, if the current speckle is found to be correlated to the reference at step 970, the current needle position is determined to be still valid.

It is desired to detect and track the needle in a fully automatic mode. However, if computation power is a limited, an alternative option is to have the user to initiate the detection by clicking a button.

The method for detecting and tracking a needle according to an embodiment of the present invention is readily realizable. It demands limited or little (with a vibrator) human intervention. Therefore, a fully automatic needle tracking and detection technique has been provided for biopsy. Moreover, since the nearby tissue instead of the needle itself is studied, the method is not sensitive to the needle position relative to the image plane, i.e., if the needle is slightly out of image plane, the method can still reliably detect the needle position.

It is to be understood, however, that even though a number of embodiments have been set forth in the foregoing description, the disclosure should not be construed to restrict the scope of the present invention, and that any equivalent changes to the structure or flow based on the present disclosure, or any direct or indirect applications to the relevant technical fields still fall into the scope of the present invention defined by the appended claims.

An embodiment of the present invention provides a method for detecting a needle, comprising arranging an ultrasound probe such that it scans an area covering a needle inserted into a tissue and nearby tissue around the needle, collecting a plurality of ultrasound frames associated with motion of the nearby tissue, determining movement information of the nearby tissue, post-processing the movement information of the nearby tissue to determine a position of the needle, and outputting information relating to the position of the needle.

According to an embodiment of the present invention, the needle is moved back and forth along a length direction of the needle to cause the motion of the nearby tissue.

In an embodiment of the present invention, the needle is moved, by hand, back and forth along the length direction. Optionally, the needle is moved, by a vibrator, back and forth along the length direction.

According to an embodiment, collecting a plurality of ultrasound frames associated with motion of the nearby tissue comprises performing a B-mode scan and collecting pulse echo data while the needle moves back and forth along the length direction.

In an embodiment of the present invention, the movement information of the nearby tissue comprises displacement and strain of the nearby tissue. Determining movement information of the nearby tissue comprises roughly estimating the position of the needle based on the displacement or strain of the nearby tissue.

In an embodiment of the present invention, roughly estimating the position of the needle based on the displacement or strain of the nearby tissue comprises determining, as the position of the needle, a position corresponding to the maximal displacement of the nearby tissue or a boundary position between positive and negative strains of the nearby tissue.

In an embodiment of the present invention, determining movement information of the nearby tissue comprises determining the displacement or strain of the nearby tissue using a Doppler method. While in an embodiment, determining movement information of the nearby tissue comprises determining the displacement or strain of the nearby tissue by performing speckle tracking between the plurality of ultrasound frames.

According to an embodiment of the present invention, the speckle tracking is implemented using one of the following algorithms: 1D or 2D cross correlation and the derivations, phase-based iterative methods, and optical flow. The speckle tracking receives, as input, detected amplitude data, beamformed RF data, or demodulated RF data.

According to an embodiment of the present invention, the aforesaid post-processing of the movement information of the nearby tissue comprises: determining the position corresponding to a maximal absolute displacement value as a peak position for each beam of a frame, removing a noise beam and an outlier beam from the beam, performing line fitting on values of the peak positions; defining a threshold for the peak values to normalize the displacement values, line smoothing the normalized displacement values, and averaging displacement values for a plurality of frames.

Optionally, post-processing the movement information of the nearby tissue further comprises upsampling an image after performing a line smoothing on the normalized displacement value.

Outputting information relating to the position of the needle comprises displaying the needle on top of an output image as a colored semi-transparent line, according to an aspect of the invention. Optionally, outputting information relating to the position of the needle comprises indicating detection states via different line colors or different line types, sign or text in a display, and verbal warning from a scanner. For example, the detection states include: “no valid detection has been made”, “valid detection is just made”, “valid detection was made and currently in tracking mode”, and “out of correlation and needle position is lost”.

According to an embodiment of the present invention, there is provided a method for tracking a needle, the method comprising arranging an ultrasound probe such that it scans an area covering a needle inserted into a tissue and nearby tissue around the needle, collecting a plurality of ultrasound frames associated with motion of the nearby tissue, determining movement information of the nearby tissue, post-processing the movement information of the nearby tissue to determine position of the needle, storing information relating to the position of the needle as a reference, determining whether the current speckle data is correlated to the reference using speckle tracking, determining the position of the needle to be lost if the current speckle data is not correlated to the reference, and determining the position of the needle to remain valid if the current speckle data is correlated to the reference.

In an embodiment, the needle is moved back and forth along a length direction of the needle to cause the motion of the nearby tissue. For example, the needle may be moved, by hand, back and forth along the length direction.

According to an embodiment of the present invention, the movement information of the nearby tissue comprises displacement and strain of the nearby tissue.

Similarly, post-processing the movement information of the nearby tissue comprises determining the position corresponding to a maximal absolute displacement value as a peak position for each beam of a frame, removing a noise beam and an outlier beam from the beam, performing line fitting on values of the peak positions, defining a threshold for the peak values to normalize the displacement values, line smoothing the normalized displacement values, and averaging displacement values for a plurality of frames.

Optionally, post-processing the movement information of the nearby tissue further comprises upsampling an image after performing a line smoothing on the normalized displacement value.

In an embodiment, the method further comprises resetting the position of the needle and/or instructing a user to poke the needle to reinitialize the detection after the position of the needle is determined to be lost.

The methods according to embodiments of the present invention are easy to operate, and do not demand extra devices. Therefore, compared to the existing techniques, the methods according to embodiments of the present invention are more cost-effective. Meanwhile, the methods according to embodiments of the present invention study not only the dynamics of the needle itself but also the dynamics of the nearby tissue, so they are equally sensitive even when the needle slightly out of the imaging plane. Therefore, embodiments of the present invention may realize higher accuracy and reliability.

What is claimed is:

1. A method for detecting a needle, comprising:
arranging an ultrasound probe such that the ultrasound probe scans an area covering a needle inserted into a tissue and nearby tissue around the needle;
collecting a plurality of ultrasound frames associated with motion of the nearby tissue;
determining movement information of the nearby tissue;
post-processing the movement information of the nearby tissue to determine a position of the needle; and
outputting information relating to the position of the needle.

2. The method according to claim 1, wherein the needle is moved back and forth along a length direction of the needle to cause the motion of the nearby tissue.

3. The method according to claim 2, wherein the needle is moved, by hand, back and forth along the length direction.

4. The method according to claim 2, wherein the needle is moved, by a vibrator, back and forth along the length direction.

5. The method according to claim 2, wherein collecting a plurality of ultrasound frames associated with motion of the nearby tissue comprises performing a B-mode scan and collecting pulse echo data while the needle moves back and forth along the length direction.

6. The method according to claim 1, wherein the movement information of the nearby tissue comprises displacement and strain of the nearby tissue.

7. The method according to claim 6, wherein determining movement information of the nearby tissue comprises roughly estimating the position of the needle based on the displacement or strain of the nearby tissue.

8. The method according to claim 7, wherein roughly estimating the position of the needle based on the displacement or strain of the nearby tissue comprises determining, as the position of the needle, a position corresponding to the maximal displacement of the nearby tissue or a boundary position between positive and negative strains of the nearby tissue.

9. The method according to claim 6, wherein determining movement information of the nearby tissue comprises determining the displacement or strain of the nearby tissue using a Doppler method.

10. The method according to claim 6, wherein determining movement information of the nearby tissue comprises determining the displacement or strain of the nearby tissue by performing speckle tracking between the plurality of ultrasound frames.

11. The method according to claim 10, wherein the speckle tracking is implemented using one of the following algorithms: 1D or 2D cross correlation and the derivations, phase-based iterative methods, and optical flow.

12. The method according to claim 10, wherein the speckle tracking receives, as input, detected amplitude data, beamformed RF data, or demodulated RF data.

13. The method according to claim 6, wherein post-processing the movement information of the nearby tissue comprises:
determining the position corresponding to a maximal absolute displacement value as a peak position for each beam of a frame;
removing a noise beam and an outlier beam from the beam;
performing line fitting on values of the peak positions;
defining a threshold for the peak values to normalize the displacement values;
line smoothing the normalized displacement values; and
averaging the displacement values for a plurality of frames.

14. The method according to claim 13, wherein post-processing the movement information of the nearby tissue further comprises upsampling an image after performing a line smoothing on the normalized displacement value.

15. The method according to claim 1, wherein outputting information relating to the position of the needle comprises displaying the needle on top of an output image as a colored semi-transparent line.

16. The method according to claim 1, wherein outputting information relating to the position of the needle comprises indicating detection states through at least one of different line colors or different line types, sign or text in a display, or verbal warning from a scanner.

17. The method according to claim 16, wherein the detection states comprise:
“no valid detection has been made”;
“valid detection is just made”;
“valid detection was made and currently in tracking mode”; and
“out of correlation and needle position is lost”.

18. A method for tracking a needle, comprising:
arranging an ultrasound probe such that the ultrasound probe scans an area covering a needle inserted into a tissue and nearby tissue around the needle;
collecting a plurality of ultrasound frames associated with motion of the nearby tissue;
determining movement information of the nearby tissue;
post-processing the movement information of the nearby tissue to determine a position of the needle;
storing information relating to the position of the needle as a reference;
determining whether current speckle data is correlated to the reference using speckle tracking;
determining the position of the needle to be lost if the current speckle data is not correlated to the reference; and
determining the position of the needle to remain valid if the current speckle data is correlated to the reference.

19. The method according to claim 18, wherein the needle is moved back and forth along a length direction of the needle to cause the motion of the nearby tissue.

20. The method according to claim 19, wherein the needle is moved, by hand, back and forth along the length direction.

21. The method according to claim 18, wherein the movement information of the nearby tissue comprises displacement and strain of the nearby tissue.

22. The method according to claim 21, wherein post-processing the movement information of the nearby tissue comprises:
determining the position corresponding to a maximal absolute displacement value as a peak position for each beam of a frame;
removing a noise beam and an outlier beam from the beam;
performing line fitting on values of the peak positions;
defining a threshold for the peak values to normalize the displacement values;
line smoothing the normalized displacement values; and
averaging the displacement values for a plurality of frames.

23. The method according to claim 22, wherein post-processing the movement information of the nearby tissue further comprises upsampling an image after performing a line smoothing on the normalized displacement value.

24. The method according to claim 18, wherein the method further comprises resetting the position of the needle and/or instructing a user to poke the needle to reinitialize the detection after the position of the needle is determined to be lost.
